(19) United States
(12) Patent Application Publication
(10) Pub. No.: US 2020/0289699 A1
(43) Pub. Date: Sep. 17, 2020

(54) L-HISTIDINE AND VITAMIN B IN OPHTHALMIC SOLUTIONS

(71) Applicant: FXS Ventures, LLC, Salem, NH (US)

(72) Inventor: Francis X. Smith, Salem, NH (US)

(21) Appl. No.: 16/351,911

(22) Filed: Mar. 13, 2019

(51) Int. Cl. A61L 12/14; C11D 3/39; C11D 3/33; C11D 3/32; C11D 3/37; C11D 3/00; C11D 3/04; C11D 3/20

(52) U.S. Cl. CPC A61L 12/142; C11D 3/3942; C11D 3/33; C11D 3/2093; C11D 3/3703; C11D 3/0078; C11D 3/046; C11D 3/32

(57) ABSTRACT

An aqueous ophthalmic solution comprising 0.00001 to about 10.0 percent by weight L-histidine, 1 ppm to 60 ppm hydrogen peroxide, 0.00001 to about 10.0 percent by weight of a B vitamin; and 0.1 to 500 parts per million of a preservative that provides superior preservative efficacy especially as against fungal microbes. These solutions may be employed in various ways including cleaning contact lenses, rinsing lenses while in the eye, storing lenses and in delivering active pharmaceutical agents to the eye.

### BACKGROUND OF THE INVENTION

The present invention relates to the field of ophthalmic solutions used to treat eyes, store contact lenses, or condition medical devices used in the eye. Such solutions are well known and widely employed with numerous products available commercially. There are several types of solutions within the field depending upon specific use. For instance, there are specific solutions for disinfecting contact lenses, solutions for cleaning contact lenses, solutions for treating the surface of contact lenses, solutions for rinsing lenses, solutions for wetting eyes, etc.

These ophthalmic solutions have been used for some period of time and are available as over the counter products. Solutions that are used in direct contact with corneal tissue such as the delivery of active pharmaceutical agent to the eye, or indirectly, such as the cleaning, conditioning or storage of devices that will come in contact with corneal tissue, such as contact lenses, there is a need to ensure that these solutions do not introduce sources of bacterial or other microbial infection. Preservatives are included to reduce the viability of microbes in the solution and to lessen the chance of contamination of the solution by the user since many of the solutions are bought, opened, used, sealed and then reused. State of the art preservative agents include polyhexamethylene biguanide (PHMB), POLYQUAD™, chlorhexidine, and benzalkonium chloride, and the like, all of which at some concentration irritate corneal tissue and lead to user discomfort. Therefore, a solution that employs a given amount of a preservative agent, but which is made more effective by addition of an agent that is not a preservative agent would be desired. The cationic preservatives are used at effective amounts as preservatives, and in the instance of PHMB from 0.0001 percent by weight to higher levels of about 0.01 weight percent.

While each of these lenses are formulated specifically for their intended application, each solution is formulated pr handled so that it will remain free of sources of infection to the eye. Numerous approaches to this problem have been employed, from methods that call for sterilization of the solution and packaging of the solution in a container that will not allow contamination. Use of specific preservative agents employed in concentrations sufficient to prevent microbial increase have been employed. Oxidative agents have been used as well as methods of irradiation. In the cases where chemical agents have been employed, there has been a tendency to employ one preservative agent in the formulation. It has been found that use of two or more specific agents in combination surprisingly provide greater efficacy in preserving solutions than state of the art single preservative systems and in particular the use of the combination of a cationic polymeric preservative, hydrogen peroxide and L-histidine provide increased preservative efficacy against fungal contamination.

This surprising effect is achievable with the further use of certain, but not all, contact lens solution agents. In particular, certain tonicity agents when employed decrease the preservative efficacy of the invention and should not be employed.

Hydrophilic lenses are particularly useful in ophthalmology due to their ability to absorb water and swell to a soft mass of good mechanical strength, and due to their transparency with the ability to retain shape and dimensions when equilibrated in ocular fluid and in storage fluids when removed from the eye.

One problem with soft contact lenses, however, is their sterilization and cleaning. The property of hydrophilic soft lenses which allows them to absorb large amounts of water also allows preservatives which might otherwise be used for cleaning and sterilization to be absorbed and later released onto the eye. The release, furthermore, may be much slower than the intake, thereby allowing preservatives to build up in the lenses. This can have the harmful result of damaging or staining contact lenses or harming the sensitive tissues of the conjunctivae or cornea.

### SUMMARY OF THE INVENTION

An aqueous ophthalmic solution comprising 0.00001 to about 10.0 percent by weight L-histidine, 1 ppm to 60 ppm hydrogen peroxide, 0.00001 to about 10.0 percent by weight of a B vitamin; and 0.1 to 500 parts per million of a preservative that provides superior preservative efficacy especially as against fungal microbes. These solutions may be employed in various ways including cleaning contact lenses, rinsing lenses while in the eye, storing lenses and in delivering active pharmaceutical agents to the eye.

In a first embodiment, a method for cleaning a contact lens is provided. The method comprising a step of cleaning the contact lens with a solution comprising: 0.00001 to about 10.0 percent by weight L-histidine; 1 ppm to 60 ppm hydrogen peroxide; 0.00001 to about 10.0 percent by weight of a B vitamin; 0.1 to 500 parts per million of a preservative; and wherein said solution has a pH greater than 5.0 and less than 8.0.

In a second embodiment, a method for storing a contact lens is provided. The method comprising a step of: soaking the contact lens in a solution for at least four hours, the solution comprising: 0.00001 to about 10.0 percent by weight L-histidine; 1 ppm to 60 ppm hydrogen peroxide; 0.00001 to about 10.0 percent by weight of a B vitamin; 0.1 to 500 parts per million of a preservative; and wherein said solution has a pH greater than 5.0 and less than 8.0.

In a third embodiment, an ophthalmic solution is provided. The solution comprising: 0.00001 to about 10.0 percent by weight L-histidine; 1 ppm to 60 ppm hydrogen peroxide; 0.00001 to about 10.0 percent by weight of a B vitamin; 0.1 to 500 parts per million of a preservative; and wherein said solution has a pH greater than 5.0 and less than 8.0.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### DETAILED DESCRIPTION OF THE INVENTION

This disclosure relates to an aqueous ophthalmic solution comprising 0.00001 to about 10.0 percent by weight histidine, 0.0001 to 3.0 percent by weight hydrogen peroxide, a select B vitamin and 0.1 to 500 parts per million of a preservative that provides superior preservative efficacy especially as against fungal microbes. These solutions may be employed in various ways including cleaning contact lenses (e.g. treating in an in vitro environment for less than five minutes), rinsing lenses while in the eye, storing lenses (i.e. soaking the lenses for at least four hours) and in delivering active pharmaceutical agents to the eye. Ophthalmic solutions are herein understood to include contact lens treatment solutions, such as cleaners, soaking solutions, conditioning solutions and lens storage solutions, as well as wetting solutions and in-eye solutions for treatment of eye conditions. The solutions may include other active ingredients useful in ophthalmic solutions such as tonicity agent, buffers, preservatives, surfactants, and antimicrobial agents.

The disclosed solutions are compatible with both rigid gas permeable and hydrophilic contact lenses and other ophthalmic devices and instruments during storage, cleaning, wetting, soaking, rinsing and disinfection. The solution can be used to deliver a pharmaceutical agent to the eye by providing the agent to the solution and then contacting the eye with the resultant solution. Alternatively, the solution can be used to clean, treat or store contact lenses by contacting the solution with the contact lens.

Histidine is a basic amino acid well known in the chemical arts and available from numerous commercial sources. Histidine is known to be used in ophthalmic ointments and the like in very concentrated forms see U.S. Pat. No. 5,811,446.

Peroxide sources may be included in the solution and are exemplified by hydrogen peroxide, and such compounds, which provide an effective resultant amount of hydrogen peroxide, such as sodium perborate decahydrate, sodium peroxide, urea peroxide and peracetic acid, an organic peroxy compound.

These B vitamins more effectively preserve solutions and reduce the degree to which cationic preservatives deposit on contact lenses. The B family of vitamins includes thiamine (B1), riboflavin (B2), niacin (B3), dexpanthenol, panthenol, pantothenic acid (B5), pyridoxine (B6), and cobalamin (B12) and salts of the aforementioned compounds. While each form of B vitamin is chemically distinct, they are often found in the same nutritional sources and hence deficiency in one is often related to deficiency in the other forms. Metabolically, they work with one another to bolster metabolism, enhance immune and nervous system function, maintain healthy skin and muscle tone, and promote cell growth and division. They may also relieve stress, depression, and cardiovascular disease. A deficiency in one B vitamin often means that intake of all B vitamins is low which is why B vitamins as a nutritional source are often provided in multivitamin or B-complex formulae.

Niacin contributes to a great number of bodily processes. Among other things niacin helps convert food into energy, build red blood cells, synthesize hormones, fatty-acids and steroids. The body uses niacin in the process of releasing energy from carbohydrates. Niacin is also needed to form fat from carbohydrates and to process alcohol. Niacin also helps regulate cholesterol.

Pyridoxine is needed to make serotonin, melatonin, and dopamine. Vitamin B-6 is an essential nutrient in the regulation of mental processes and possibly assists in mood and many other health concerns Cobalamin is needed for normal nerve cell activity. Vitamin B-12 is also needed for DNA replication, and production of the mood-affecting substance called SAMe (S-adenosyl-L-methionine). Vitamin B-12 works with folic acid to control homocysteine levels. An excess of homocysteine, which is an amino acid (protein building block), may increase the risk of heart disease, stroke, and perhaps osteoporosis and Alzheimer's disease.

Pantothenic acid, also sometimes referred to as coenzyme A, is the physiologically active form of pantothenic acid, and serves a vital role in metabolism as a coenzyme for a variety of enzyme-catalyzed reactions involving transfer of acetyl (two-carbon) groups. Surprisingly, pantothenic acid is essential for the growth of various microorganisms, including many strains of pathogenic bacteria.

Other compounds such as folic acid or folate are active in combination with the B vitamins and are needed to synthesize DNA. DNA allows cells to replicate normally. In some embodiments the solutions described herein further comprise folic acid or folate in a concentration between 0.001 and 10.0 weight percent. Folic acid is especially important for the cells of a fetus when a woman is pregnant. Folic Acid is also needed to make SAMe and keep homocysteine levels in the blood from rising. Folic Acid (pteroylglutamic acid) is not active as such in the mammalian organism, but rather is enzymatically reduced to tetrahydrofolic acid (THFA), the coenzyme form. An interrelationship exists with vitamin B12 and folate metabolism that further involves vitamin B6: folate coenzymes participate in a large number of metabolic reactions in which there is a transfer of a one-carbon unit.

The cationic polymeric preservative includes polymeric biguanides such as polymeric hexamethylene biguanides (PHMB), and combinations thereof. Such cationic polymeric biguanides, and water-soluble salts thereof, having the following formula:

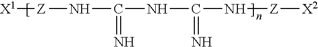

wherein Z is an organic divalent bridging group which may be the same or different throughout the polymer, n is on average at least 3, preferably on average 5 to 20, and X¹ and X² are

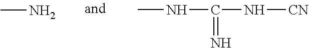

One preferred group of water-soluble polymeric biguanides will have number average molecular weights of at least 1,000 and more preferably will have number average molecular weights from 1,000 to 50,000. Suitable water-soluble salts of the free bases include, but are not limited to hydrochloride, borate, acetate, gluconate, sulfonate, tartrate and citrate salts.

Most preferred are the polymeric hexamethylene biguanides, commercially available, for example, as the hydrochloride salt from Zeneca (Wilmington, Del.) under the trademark COSMOCIL™ CQ. Such polymers and water-soluble salts are referred to as polyhexamethylene (PHMB) or polyaminoptopyl biguanide (PAPB). The term polyhexamethylene biguanide, as used herein, is meant to encompass one or more biguanides have the following formula:

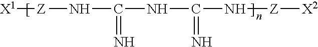

wherein Z, X¹ and X² are as defined above and n is from 1 to 500.

Depending on the manner in which the biguanides are prepared, the predominant compound falling within the above formula may have different X¹ and X² groups or the same groups, with lesser amounts of other compounds within the formula. Such compounds are known and are disclosed in U.S. Pat. No. 4,758,595 and British Patent 1,432,345, which patents are hereby incorporated. Preferably, the water-soluble salts are compounds where n has an average value of 2 to 15, most preferably 3 to 12.

In another embodiment, a polymeric biguanide is used in combination with a bis(biguanide) compound. Polymeric biguanides, in combination with bisbiguanides such as alexidine, are effective in concentrations as low as 0.00001 weight percent (0.1 ppm). It has also been found that the bactericidal activity of the solutions may be enhanced or the spectrum of activity broadened through the use of a combination of such polymeric biguanides with alexidine or similar biguanides.

An optional non-biguanide disinfectant/germicide can be employed as a solution preservative, but it may also function to potentiate, complement or broaden the spectrum of microbiocidal activity of another germicide. This includes microbiocidally effective amounts of germicides which are compatible with and do not precipitate in the solution, in concentrations ranging from about 0.00001 to about 0.5 weight percent, and more preferably, from about 0.0001 to about 0.1 weight percent. Suitable complementary germicidal agents include, but are not limited to, quaternary ammonium compounds or polymers, thimerosal or other phenylmercuric salts, sorbic acid, alkyl triethanolamines, and mixtures thereof. Representative examples of the quaternary ammonium compounds are compositions comprised of benzalkonium halides or, for example, balanced mixtures of n-alkyl dimethyl benzyl ammonium chlorides. Other examples include polymeric quaternary ammonium salts used in ophthalmic applications such as poly[(dimedhyliminio)-2-butene-1,4-diyl chloride], [4-tris(2-hydroxyethyl) ammonio]-2-butenyl-w-[tris(2-hydroxyethyl)ammonio]dichloride (chemical registry number 75345-27-6) generally available as polyquaternium I (r) from ONYX Corporation, or those described in U.S. Pat. No. 6,153,568.

The additional preservatives useful with the disclosed solutions are known, such as N-alkyl-2-pyrrolidone, polyquaternium-1, hexetidine, bronopol are also known.

The disclosed solutions may further contain other additives including but not limited to buffers, tonicity agents, demulcents, wetting agents, sequestering agents (chelating agents), surface active agents, viscosity inducing agents and enzymes. In one embodiment between about 0.01% and 5.0% by weight of a simple saccharide is present. Examples of simple saccharides include mannitol; sorbitol; sucrose; dextrose and glycerin.

The pH of the solutions should be adjusted to be compatible with the eye and the contact lens, such as between 6.0 to 8.0, preferably between 6.8 to 7.8 or between 7.0 to 7.6. Significant deviations from neutral (pH 7.3) will cause changes in the physical parameters (i.e. diameter) in some contact lenses. Low pH (pH less than 5.5) can cause burning and stinging of the eyes, while very low or very high pH (less than 3.0 or greater than 10) can cause ocular damage.

Suitable buffers may be added, such as boric acid, sodium borate, potassium citrate, citric acid, sodium bicarbonate, and various mixed phosphate buffers (including combinations of Na₂HPO₄, NaH₂PO₄ and KH₂PO₄) and mixtures thereof. Borate buffers may be used, particularly for enhancing the efficacy of PAPB. Generally, buffers will be used in amounts ranging from about 0.05 to 2.5 percent by weight, and preferably, from 0.1 to 1.5 percent. The formulations may also include buffers such as phosphates, bicarbonate, citrate, borate, ACES, BES, BICINE, BIS-Tris Propane, HEPES, HEPPS, imidazole, MES, MOPS, PIPES, TAPS, TES, TRIS and Tricine.

Ophthalmologically acceptable chelating agents useful in the present invention include amino carboxylic acid compounds or water-soluble salts thereof, including ethylenediaminetetraacetic acid, nitrilotriacetic acid, diethylenetriamine pentaacetic acid, hydroxyethylethylenediaminetriacetic acid, 1,2-diaminocyclohexanetetraacetic acid, ethylene glycol bis (beta-aminoethyl ether) in N, N, N′, N′ tetraacetic acid (EGTA), aminodiacetic acid and hydroxyethylamino diacetic acid. These acids can be used in the form of their water-soluble salts, particularly their alkali metal salts. Especially preferred chelating agents are the di-, tri- and tetra-sodium salts of ethylenediaminetetraacetic acid (EDTA), most preferably disodium EDTA (Disodium Edetate).

Other chelating agents such as citrates and polyphosphates can also be used in the present invention. The citrates which can be used in the present invention include citric acid and its mono-, di-, and tri-alkaline metal salts. The polyphosphates which can be used include pyrophosphates, triphosphates, tetraphosphates, trimetaphosphates, tetrametaphosphates, as well as more highly condensed phosphates in the form of the neutral or acidic alkali metal salts such as the sodium and potassium salts as well as the ammonium salt.

Suitable tonicity agents include sodium chloride, potassium chloride, glycerol or mixtures thereof. The tonicity of the solution is typically adjusted to approximately 240-310 milliosmoles per kilogram solution (mOsm/kg) to render the solution compatible with ocular tissue and with hydrophilic contact lenses. In one embodiment, the solution contains 0.01 to 0.35 weight percent sodium chloride. The important factor is to keep the concentrations of such additives to a degree no greater than that would supply a chloride concentration of no greater than about 0.2 mole percent.

The solutions may also include surfactants such as a polyoxyethylene-polyoxypropylene nonionic surfactant which, for example, can be selected from the group of commercially available surfactants having the name poloxamine or poloxamer, as adopted by the CTFA International Cosmetic Ingredient Dictionary. The poloxamine surfactants consist of a poly(oxypropylene)-poly(oxyethylene) adduct of ethylene diamine having a molecular weight from about 7,500 to about 27,000 wherein at least 40 weight percent of said adduct is poly(oxyethylene), has been found to be particularly advantageous for use in conditioning contact lenses when used in amounts from about 0.01 to about 15 weight percent. Such surfactants are available from BASF Wyandotte Corp., Wyandotte, Mich., under the registered trademark TETRONIC®. The poloxamers are an analogous series of surfactants and are polyoxyethylene, polyoxypropylene block polymers available from BASF Wyandotte Corp., Parsippany, N.J. 07054 under the trademark PLURONIC™.

Surfactants that might be employed include polysorbate surfactants, polyoxyethylene surfactants, phosphonates, saponins and polyethoxylated castor oils, but preferably the polyethoxylated castor oils. These surfactants are commercially available. The polyethoxylated castor oils are sold by BASF under the trademark CREMOPHOR™.

The HLB of a surfactant is known to be a factor in determining the emulsification characteristics of a nonionic surfactant. In general, surfactants with lower HLB values are more lipophilic, while surfactants with higher HLB values are more hydrophilic. The HLB values of various poloxamines and poloxamers are provided by BASE Wyandotte Corp., Wyandotte, Mick Preferably, the HLB of the surfactant in the present invention is at least 18, more preferably 18 to 32, based on values reported by BASF.

Additional compatible surfactants that are known to be useful in contact wetting or rewetting solutions can be used in the solutions of this invention. The surfactant should be soluble in the lens care solution and non-irritating to eye tissues. Satisfactory non-ionic surfactants include polyethylene glycol esters of fatty acids, e.g. coconut, polysorbate, polyoxyethylene or polyoxypropylene ethers of higher alkanes (C₁₂-C₁₈). Examples of the preferred class include polysorbate 20 (available from ICI Americas Inc., Wilmington, Del. 19897 under the trademark TWEEN® 20), polyoxyethylene (23) lauryl ether (BRIJ® 35), polyoxyethylene (40) stearate (MYRJ® 52), polyoxyethylene (25) propylene glycol stearate (ATLAS® G 2612). BRIJ® 35, MYRJ® 52 and ATLAS® G 2612 are trademarks of, and are commercially available from, ICI Americas Inc., Wilmington, Del. 19897.

Various other surfactants suitable can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C. however, the preferred surfactants are commercially available surfactants sold under the trademark CREMAPHOR™ RH40® by BASF which are polyoxyethoxylated castor oils.

Suitable viscosity inducing agents can include lecithin or the cellulose derivatives such as hydroxyethylcellulose, hydroxypropylmethylcellulose and methylcellulose in amounts similar to those for surfactants, above.

In one embodiment, the solution comprises 0.00001 to 10.0 percent by weight histidine, between 1 ppm and 60 ppm hydrogen peroxide, 0.1 to 500 parts per million of a preservative, a hydrogenated castor oil and between 0.00001 and 10.0 weight percent of a B vitamin. In one such embodiment, the solution is used in an in vitro environment to clean a contact lens.

### EXAMPLES

The following examples illustrate the inventor but do not fully delineate the scope of the invention intended by the inventor to be claimed herein. They are intended to illustrate how the invention might be practiced in certain particulars, but are not meant to be interpreted by those of skill in this art restrictively.

### Example 1 Histidine-Peroxide

Formulations were prepared by dissolving L-histidine in water. The pH of the solutions were adjusted to 7.3 with 1N hydrochloric acid. Hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two-hour exposure. The activity is expressed as a log reduction from the initial inoculum. The compositions, concentrations and activity of each of the solutions are summarized in the following table.

| Log | | | Hydrogen | |
|---|---|---|---|---|
| Reduction | Preservative | Buffer | Peroxide | Dequest 2010 |
| 1.25 | PHMB 0.0001% | L-histidine 0.2% | None | 0.006% |
| 1.85 | PHMB 0.0001% | L-histidine 0.2% | 0.006% | 0.006% |

The results demonstrate the improved antifungal efficacy of the histidine-hydrogen peroxide combination against C. albicans.

### Example 2—Histidine-Peroxide

Formulations were prepared by dissolving L-histidine in water. The pH of the solutions were adjusted to 7.3 with 1N hydrochloric acid. Sodium chloride, Hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two-hour exposure. The activity is expressed as a log reduction from the initial inoculum. The compositions, concentrations and activity of each of the solutions are summarized in the following table.

| Log | | | Sodium | Hydrogen | Dequest |
|---|---|---|---|---|---|
| Reduction | Preservative | Buffer | Chloride | Peroxide | 2010 |
| 0.5 | PHMB 0.0001% | L-histidine 0.2% | 0.4% | None | 0.006% |
| 1.08 | PHMB 0.0001% | L-histidine 0.2% | 0.4% | 0.006% | 0.006% |

The results demonstrate the improved antifungal efficacy of the histidine-hydrogen peroxide combination against C. albicans.

### Example 3—Histidine-Peroxide

Formulations were prepared by dissolving L-histidine in water. The pH of the solutions were adjusted to 7.3 with 1N hydrochloric acid. Glycerin, hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| Log | | | | Hydrogen | Dequest |
|---|---|---|---|---|---|
| Reduction | Preservative | Buffer | Glycerine | Peroxide | 2010 |
| 1.60 | PHMB 0.0001% | L-histidine | None | None | None |
| | | 0.2% | | | |
| 1.08 | PHMB 0.0001% | L-histidine | None | 0.006% | None |
| | | 0.2% | | | |
| 1.27 | PHMB 0.0001% | L-histidine | None | None | 0.006% |
| | | 0.2% | | | |
| 2.25 | PHMB 0.0001% | L-histidine | None | 0.006% | 0.006% |
| | | 0.2% | | | |
| 1.08 | PHMB 0.0001% | L-histidine | None | None | 0.003% |
| | | 0.2% | | | |
| 2.04 | PHMB 0.0001% | L-histidine | None | 0.006% | 0.003% |
| | | 0.2% | | | |
| 1.57 | PHMB 0.0001% | L-histidine | 0.50% | None | None |
| | | 0.2% | | | |
| 2.15 | PHMB 0.0001% | L-histidine | 0.50% | 0.006% | None |
| | | 0.2% | | | |
| 1.25 | PHMB 0.0001% | L-histidine | 0.50% | None | 0.006% |
| | | 0.2% | | | |
| 2.04 | PHMB 0.0001% | L-histidine | 0.50% | 0.006% | 0.006% |
| | | 0.2% | | | |
| 1.08 | PHMB 0.0001% | L-histidine | 0.50% | None | 0.003% |
| | | 0.2% | | | |
| 1.93 | PHMB 0.0001% | L-histidine | 0.50% | 0.006% | 0.003% |
| | | 0.2% | | | |

The results demonstrate the improved antifungal against C. albicans in each paired formulation, when 0.006% hydrogen peroxide is added. The data demonstrates that the increased activity is independent of the presence of Dequest 2010.

### Example 4—Histidine-Peroxide

Formulations were prepared by dissolving L-histidine in water. The pH of the solutions were adjusted to 7.3 with 1N hydrochloric acid. Hydrogen peroxide, Dequest 2010 and polyhexamethylenebiguanide HCl (PHhMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| Log | | | Hydrogen | Dequest |
|---|---|---|---|---|
| Reduction | Preservative | Buffer | Peroxide | 2010 |
| 2.01 | PHMB 0.0001% | Histidine 0.2% | None | None |
| 2.42 | PHMB 0.0001% | Histidine 0.2% | 0.006% | 0.003% |

| | |
|---|---|
| 0.73 | Marketed Product * |
| 1.95 | Marketed product 2 ** |

The results demonstrate the improved antifungal efficacy of the histidine-hydrogen peroxide combination. The effectiveness was superior to that found in either commercially marketed products.

### Example 5—Histidine-Peroxide

Formulations were prepared by dissolving L-histidine in water, The pH of the solutions were adjusted to 7.3 with 1N hydrochloric acid. CREMOPHOR RH40™ (polyoxyl 40 hydrogenated castor oil), hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| Log | | | | Hydrogen | Dequest |
|---|---|---|---|---|---|
| Reduction | Preservative | Buffer | Additive | Peroxide | 2010 |
| 2.51 | PHMB | L-histidine 0.2% | CREMOPHOR | None | None |
| | 0.0001% | | RH40 ™ | | |
| 3.27 | PHMB | L-histidine 0.2% | CREMOPHOR | 0.006% | 0.003% |
| | 0.0001% | | RH40 ™ | | |

*marketed product 1 having the general composition: A sterile isotonic aqueous solution containing sodium chloride, polyoxyethylene polyoxypropylene block copolymer, sodium phosphate dibasic, sodium phosphate monobasic, and preserved with edetate disodium dihydrate 0.025% and polyhexanide 0.0001%.
**marketed product 2 having the general composition: A sterile, isotonic solution that contains HYDRANATE (hydroxyalkylphosphonate), boric acid. edetate disodium, poloxamine, sodium borate and sodium chloride; preserved with DYMED (polyaminopropyl biquanide) 0.0001%.

The results demonstrate the improved antifungal efficacy of the histidine-hydrogen peroxide combination against C. albicans.

### Example 6—Histidine-Peroxide

Formulations were prepared by dissolving L-histidine in water. The pH of the solutions were adjusted to 7.3 with 1N hydrochloric acid. The tonicity agent, hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| Log | | | Tonicity | Wetting | Hydrogen | Dequest |
|---|---|---|---|---|---|---|
| Red. | Preservative | Buffer | Agent | Agent | Peroxide | 2010 |
| 2.42 | PHMB | L- | None | CREMOPHOR | | |
| | 0.0001% | histidine | | RH 40 ™ | | |
| | | 0.2% | | | | |
| 3.34 | PHMB | L- | None | CREMOPHOR | 0.006% | 0.003% |
| | 0.0001% | histidine | | RH 40 ™ | | |
| | | 0.2% | | | | |
| 2.19 | PHMB | L- | Glycerin | CREMOPHOR | | |
| | 0.0001% | histidine | 3% | RH 40 ™ | | |
| | | 0.2% | | | | |
| 2.94 | PHMB | L- | Glycerin | CREMOPHOR | 0.006% | 0.003% |
| | 0.0001% | histidine | 3% | RH 40 ™ | | |
| | | 0.2% | | | | |
| 2.19 | PHMB | L- | Propylene | CREMOPHOR | | |
| | 0.0001% | histidine | glycol | RH 40 ™ | | |
| | | 0.2% | 3% | | | |
| 2.95 | PHMB | L- | Propylene | CREMOPHOR | 0.006% | 0.003% |
| | 0.0001% | histidine | glycol | RH 40 ™ | | |
| | | 0.2% | 3% | | | |
| 3.36 | PHMB | L- | Sorbitol | CREMOPHOR | | |
| | 0.0001% | histidine | 5% | RH 40 ™ | | |
| | | 0.2% | | | | |
| 3.92 | PHMB | L- | Sorbitol | CREMOPHOR | 0.006% | 0.003% |
| | 0.0001% | histidine | 5% | RH 40 ™ | | |
| | | 0.2% | | | | |

| Log | | Hydrogen | Dequest | |
|---|---|---|---|---|
| Reduction | Preservative | Buffer | Peroxide | 2010 |

| | |
|---|---|
| 0.68 | Marketed Product 1 * |
| 2.99 | Marketed Product 2 ** |
| 2.98 | Marketed Product 3 (Opti-Free Express) |

* marketed product 1 having the general composition: A sterile isotonic aqueous solution containing sodium chloride, polyoxyethylene polyoxypropylene block copolymer, sodium phosphate dibasic, sodium phosphate monobasic, and preserved with edetate disodium dihydrate 0.025% and polyhexanide 0.0001%.
** marketed product 2 having the general composition: A sterile, isotonic solution that contains HYDRANATE (hydroxyalkylphosphonate), boric acid, edetate disodium, poloxamine, sodium borate and sodium chloride; preserved with DYMED (polyaminopropyl biquanide) 0.0001%.

The data shows that the addition of 0.006% hydrogen peroxide to histidine provides increased antifungal activity against C. albicans. Consistent results were found in the presence of CREMOPHOR RH40™ with glycerin, propylene glycol, and soribitol. All formulations with dilute hydrogen peroxide added to histidine were equal to or superior to marketed products.

### Example 7

Formulations containing Pyridoxine HCl (Spectrum) and Thiamine HCl (Fisher) were prepared in a 0.2% phosphate buffer. The solutions were made isotonic with sodium chloride and preserved with polyhexamethylene biguanide at 0.0001%. The pH was adjusted to 7.2 with either 1 N sodium hydroxide or 1 N hydrochloric acid. The in vitro microbicidal activity of the solutions was determined by exposing C. albicans to 10 ml of each solution at room temperature for 4 hours. Subsequently, an aliquot of each solution was serial diluted onto agar plates and incubated for 48 hours at elevated temperatures. At the conclusion of the incubation period the plates are examined for the development of colonies. The log reduction was determined based on a comparison to the inoculum control. The following table provides the results of the in vitro studies.

| Additive | 4 Hour Log Reduction |
|---|---|
| Pyridoxine HCl (0.5%) | 2.0 |
| Thiamine HCl (0.5%) | 1.0 |
| Buffer Control | 0.8 |

The solution containing pyridoxine HCl and thiamine HCl showed an improvement in the activity against C. albicans as compared to the buffer control.

### Example 8

Formulations containing dexpanthenol were prepared in a 0.25% Bis-Tris Propane buffer. The solutions were made isotonic with sodium chloride and preserved with polyhexamethylene biquanide at 0.00005%. The pH was adjusted to 7.2 with either 1 N sodium hydroxide or 1 N hydrochloric acid. The in vitro microbicidal activity of the solutions was determined by exposing C. albicans to 10 ml of each solution at room temperature for 4 hours. Subsequently, an aliquot of each solution was serial diluted onto agar plates and incubated for 48 hours at elevated temperatures. At the conclusion of the incubation period the plates are examined for the development of colonies. The log reduction was determined based on a comparison to the inoculum control. The following table provides the results of the in vitro studies.

| | | | | Log Reduction |
|---|---|---|---|---|
| | | | | Calibicans 4 |
| Buffer | Additive | Preservative | Dexpanthenol | hour |
| Bis-Tris | Cremophor | PHMB 0.5 | None | 3.8 |
| Propane 0.25% | 0.20% | ppm | | |
| | Inositol 3.0% | | | |
| | Allantoin 0.1% | | | |
| Bis-Tris | Cremophor | PHMB 0.5 | Dexpanthenol | 4.9 |
| Propane 0.25% | 0.20% | ppm | 0.1% | |
| | Inositol 3.0% | | | |
| | Allantoin 0.1% | | | |

This data shows that the dexpanthenol has improved preservative efficacy over a solution with a preservative alone.

### Example 9

Formulations containing Dexpanthanol (Spectrum), Pyridoxine HCl (Spectrum) Thiamine HCl (Spectrum), and no Vitamin B control were prepared in a 0.5% Tris buffer containing 0.6% sodium chloride. The pH was adjusted with 1 N HCl to a final pH of 7.2. Polyhexamethylene biquanide (PHMB) at 0.0001% was added to each formulation. The in vitro anti-microbial activity of the solutions was determined by exposing E. coli to 10 ml of each solution at room temperature for 1 hours. Subsequently, an aliquot of each solution was serial diluted onto agar plates and incubated for 48 hours at elevated temperatures. At the conclusion of the incubation period the plates are examined for the development of colonies. The log reduction was determined based on a comparison to the inoculum control. The following table provides the results of the in vitro studies.

| | | | Log Reduction |
|---|---|---|---|
| Solution | | | at 1 hr |
| 0.5% Dexpanthenol | 0.5% Tris | 1 ppm PHMB | 4.82 |
| 0.5% Pyridoxine | 0.5% Tris | 1 ppm PHMB | 4.34 |
| HCl | | | |
| 0.5% Thiamine HCl | 0.5% Tris | 1 ppm PHMB | 5.12 |
| None | 0.5% Tris | 1 ppm PHMB | 0.42 |

The results showed an enhancement of the preservative in the presence of the Dexpanthanol, Pyridoxine, and Thiamine.

### Example 10

Formulations containing Dexpanthanol (Spectrum), Pyridoxine HCl (Spectrum) Thiamine HCl (Spectrum), and no Vitamin B control were prepared in a 0.5% Tris buffer containing 0.6% sodium chloride. The pH was adjusted with 1 N HCl to a final pH of 7.2. Benzalkonium Chloride (BAK) at 0.0025% was added to each formulation. The in vitro anti-microbial activity of the solutions was determined by exposing E. coli to 10 ml of each solution at room temperature for 1 hour. Subsequently, an aliquot of each solution was serial diluted onto agar plates and incubated for 48 hours at elevated temperatures. At the conclusion of the incubation period the plates are examined for the development of colonies. The log reduction was determined based on a comparison to the inoculum control. The following table provides the results of the in vitro studies.

| | | | | Log Reduction |
|---|---|---|---|---|
| Solution | | | | at 1 hr |
| 0.5% Dexpanthenol | 0.5% Tris | 25 | PPM BAK | >5.12 |
| 0.5% Pyridoxine | 0.5% Tris | 25 | PPM BAK | >5.12 |
| HCl | | | | |
| 0.5% Thiamine HCl | 0.5% Tris | 25 | PPM BAK | 3.30 |
| None | 0.5% Tris | 1 | ppm PHMB | 0.42 |

The results showed an enhancement of the preservative in the presence of the Dexpanthanol and pyridoxine.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for cleaning a contact lens comprising a step of:
cleaning the contact lens with a solution comprising:
0.00001 to about 10.0 percent by weight L-histidine;
1 ppm to 60 ppm hydrogen peroxide;
0.00001 to about 10.0 percent by weight of a B vitamin;
0.1 to 500 parts per million of a preservative; and
wherein said solution has a pH greater than 5.0 and less than 8.0.

2. The method as recited in claim 1, wherein the preservative is a polymeric preservative of the formula: 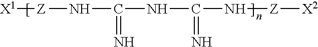
wherein Z is an organic divalent bridging group, n is at least 3, and X¹ and X² are:

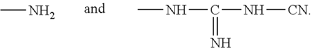

3. The method as recited in claim 2, wherein the polymeric preservative is polymeric hexamethylene biguanide.

4. The method as recited in claim 1, wherein the solution further comprises from 0.05 to 2.5 wt % of a buffer.

5. The method as recited in claim 4, wherein the buffer is selected from the group consisting of boric acid, sodium borate, potassium citrate, citric acid, sodium bicarbonate, bis-tris propane, TRIS, mixed phosphate buffers and mixtures thereof.

6. The method as recited in claim 1, wherein the solution further comprises a chelating agent.

7. The method as recited in claim 6, wherein the chelating agent is selected from the group consisting of: ethylenediaminetetraacetic acid, nitrilotriacetic acid, diethylenetriamine pentaacetic acid, hydroxyethylethylenediaminetriacetic acid, 1,2-diaminocyclohexanetetraacetic acid, ethylene glycol bis (beta-aminoethyl ether) in N, N, N′, N′ tetraacetic acid (EGTA), aminodiacetic acid, hydroxyethylamino diacetic acid, ethylenediaminetetraacetic acid (EDTA) and disodium Edetate.

8. The method as recited in claim 1, wherein the solution further comprises 0.01 to 0.35 wt % sodium chloride.

9. The method as recited in claim 1, wherein the B vitamin is selected from a group consisting of thiamine (B1), riboflavin (B2), niacin (B3), dexpanthenol, panthenol, pantothenic acid (B5), pyridoxine (B6), and cobalamin (B12).

10. The method as recited in claim 1, wherein the B vitamin is dexpanthenol.

11. The method as recited in claim 1, wherein the solution further comprises a polyoxyl 40 hydrogenated castor oil;

12. A method for storing a contact lens comprising a step of:
soaking the contact lens in a solution for at least four hours, the solution comprising:
0.00001 to about 10.0 percent by weight L-histidine;
1 ppm to 60 ppm hydrogen peroxide;
0.00001 to about 10.0 percent by weight of a B vitamin;
0.1 to 500 parts per million of a preservative; and
wherein said solution has a pH greater than 5.0 and less than 8.0.

13. The method as recited in claim 12, wherein the B vitamin is selected from a group consisting of thiamine (B1), riboflavin (B2), niacin (B3), dexpanthenol, panthenol, pantothenic acid (B5), pyridoxine (B6), and cobalamin (B12).

14. The method as recited in claim 12, wherein the B vitamin is dexpanthenol.

15. An ophthalmic solution comprising:
0.00001 to about 10.0 percent by weight L-histidine;
1 ppm to 60 ppm hydrogen peroxide;
0.00001 to about 10.0 percent by weight of a B vitamin;
0.1 to 500 parts per million of a preservative; and
wherein said solution has a pH greater than 5.0 and less than 8.0.
